(19) United States
(12) Patent Application Publication
(10) Pub. No.: US 2009/0246288 A1
(43) Pub. Date: Oct. 1, 2009

(54) Taste-masking oral dosage form and method of preparing the same

(75) Inventors: Chih-Chiang Yang, Taipei City (TW); Wen-Che Wang, Danshuei Township (TW); Hui-Yu Chen, Banciao City (TW)

Correspondence Address: ANDREWS KURTH LLP, 1350 I STREET, N.W., SUITE 1100, WASHINGTON, DC, 20005, US

(73) Assignee: Pharmaceutical Industry Technology and Development Center

(21) Appl. No.: 12/457,151

(22) Filed: Jun. 2, 2009

Related U.S. Application Data
(63) Continuation-in-part of application No. 10/839,243, filed on May 6, 2004.

(30) Foreign Application Priority Data
Feb. 25, 2004 (TW) 93104745

(51) Int. Cl. A61K 9/14; A61K 47/26; A61K 31/167; A61K 9/00

(52) U.S. Cl. 424/499; 514/778; 514/629; 424/400

(57) ABSTRACT

The present invention provides a taste-masking oral dosage form which comprises a taste-masked microparticle and an excipient. The taste-masked microparticle comprises a taste-masked crystal containing an active pharmaceutical ingredient masked by a hydrophilic polymer and a starch. The present invention also provides a method for making the taste-masked microparticles and taste-masking oral dosage form.

### 1. BACKGROUND OF THE INVENTION

a. Field of the Invention

The present invention relates to a pharmaceutical composition, and more specifically to a taste-masking oral dosage form and method of preparing the same.

b. Description of the Related Art

There are various types of oral administrative medicines, such as tablets, capsules, granules, powders, syrups and the like. Orally administrated medicines, however, suffer from many drawbacks. For example, active pharmaceutical ingredients (“APIs”) in medicines may leave an unpleasant taste after drug administration.

Tablets and capsules, for example, may be hard to swallow for the elderly or children. Granules and powders may possibly enter the respiratory tract or lungs. Additionally, dosage of syrups, may be difficult measured, particularly for the elderly or children.

Therefore, many researches regarding taste-masking oral dosage forms have been undertaken recently to provide a new generation rapidly dissolved and safely swallowed tablets, and their dosages can be accurately measured, particularly can mask bitter. Additionally, tablets should possess adequate hardness to facilitate the packaging process.

Several related arts are disclosed in the following. U.S. Pat. No. 5,804,212 describes a preparation in which a starch and a nasal drug are blended to form a microparticle to improve nasal absorption. European Patent No. 230264 discloses an aqueous nasal drug delivery system for vaccines comprising a two examples (U.S. Pat. No. 5,804,212 and European Patent No. 230264) merely cite how to improve drug absorption effects, without commenting on taste-masking effects.

Japanese Patent Laid-open No. 76420/1977 and 24410/1983 describe a method of preparing a porous tablet which comprises blending a tablet-constituting composition with inert solvent, solidifying, compressing the resulting solid into tablets, and evaporating solvent by freeze-drying. U.S. Pat. No. 5,501,861 discloses a method of preparing a fast dissolving tablet comprising a water-soluble saccharide (such as sugar, starch, lactose, sugar alcohol, or tetrose) and a pharmacologically active ingredient, which comprises compressing the blended solid into tablets with a molding pressure of 5˜130 kg/cm² and evaporating the solvent by freeze-drying.

The above fast dissolving tablets are prepared by Zydis (from R. P. Scherer, England) freeze-drying. This method, however, suffer from high process cost and insufficient mechanical strength of the preparation.

Therefore, it is necessary to develop a preparation which offers a pleasant taste and acceptable disintegration speed in an oral cavity after dosage, and possesses a sufficient mechanical strength so as to protect the preparation from destruction in the course of manufacture.

### 2. SUMMARY OF THE INVENTION

In order to solve the conventional problems, an object of the invention is to provide an oral dosage form having taste-masking effects, rapid disintegration rates, sufficient hardness to resist destruction during the course of manufacture and storage, and low cost.

In one embodiment of the invention, a taste-masked microparticle which comprises (a) an active pharmaceutical ingredient crystal masked by a hydrophilic polymer to form a taste-masked API crystal and (2) a starch, is provided. The taste-masked microparticle comprises ≧95% by weight of the taste-masked API crystal. The API is an orally administered drug, preferably with bitter taste. The hydrophilic polymer is at least one selected from group consisting of polyethylene glycol (PEG), PVP, carbopol, polysaccharide, agar, MC, and HPMC, preferably PEG, and most favorably PEG 6000.

The taste-masked API crystal is morphologically different from the API crystal. For example, a Taste-masked API crystal made of acetaminophen as API and PEG 6000 as hydrophilic polymer converts a tubular shaped API into a polyhedral shaped crystal. In addition, the bitter taste associated with acetaminophen does not appear in the taste-masked API crystal due to the masking of the API by the hydrophilic polymer.

The starch is at least one selected from the group consisting of amylodextrin, hydroxyethyl starch, hydropropyl starch, carboxymethyl starch, acetylated starch, and phosphorylated starch.

The microparticle has a diameter in the range between about 150 μm and about 350 μm.

In another embodiment of the invention, a taste-masking oral dosage form is provided. The taste-masking oral dosage form contains a taste-masked microparticle and an excipient. The taste-masked microparticle comprises an API masked by a hydrophilic polymer and a starch. The taste-masking oral dosage form is preferably a tablet, and further preferably contains a surfactant.

The tablet provided in the invention may be rapidly dissolved in an oral cavity, due to the hydrophilic polymer having strong water absorption, so that it can be advantageously used for treatment of diseases in the elderly or children. Additionally, the dissolution rate of the tablet is improved by the surfactant in an oral cavity, particularly for very slightly dissolved drugs.

In another embodiment of the invention, a taste-masked API is provided. The taste-masked API comprises an API with a bitter taste and a hydrophilic polymer, wherein the hydrophilic polymer masks the bitter taste of the API.

In yet another embodiment of the invention, a method for preparing a taste-masking oral dosage form is provided. The method comprises the following steps. First, a first solution is provided which comprises an active pharmaceutical ingredient (“API”) and a starch dissolved in aqueous solution, preferably in water or alcohol. Second, a second solution is provided which comprises a hydrophilic polymer and optionally a surfactant. The first solution is slowly added into the second solutions with continuous stirring to form a plurality of the taste-masked microparticles. It is preferred that the first solution be heated to about or above 85° C. to facilitate the dissolution of the API. It is also preferably to heat the second solution to about or above 60° C. to dissolve the hydrophilic polymer and optionally the surfactant. It is noted that no crystals and/or microparticles is sedimented in the first or the second solutions, respectively. However, after the mixing of the first and the second solutions and particularly when the temperature of the mixed solutions has dropped down to about or below room temperature, the microparticles, which contain the taste-masked API crystals packaged with the starch (i.e., forming individual microspheres) begin to sediment. The collected microparticles can be further compressed through a compression-molding process to form a tablet.

The tablet has an adequate hardness and rapid dissolving rate, due to the specific granulation and additives thereof. More particularly, the bitter taste is masked, due to the masking of the API by the hydrophilic polymer and/or the packaging of the microparticles with the starch microspheres. Additionally, the low-cost compression-molding process satisfies industry requirements.

A detailed description is given in the following embodiments with reference to the accompanying drawings.

### 3. BRIEF DESCRIPTION OF THE DRAWINGS

The present invention can be more fully understood by reading the subsequent detailed description and examples with references made to the accompanying drawings, wherein:

FIG. 1 is a flow chart of the granulating process of the invention.

FIG. 2 shows the microparticle structure prepared by the invention.

FIG. 3 is a flow chart of the compression-molding process of the invention.

FIGS. 4A-4C are microscopic pictures showing crystals of acetaminophen (FIG. 4A), PEG 6000 (FIG. 4B) and starch (FIG. 4C), respectively, before they were dissolved in the first and the second solution, respectively.

FIG. 5 is a microscopic picture showing the starch microspheres formed by mixing a heated first solution containing only starch with a heated second solution containing only PEG 6000. When the temperature of the mixed solutions dropped below room temperature under continuous stirring, the resulting starch microspheres were formed.

FIG. 6 is a microscopic picture of the taste-masking microparticles formed by mixing a heated first solution of acetaminophen and starch with a heated second solution of PEG 6000. When the temperature of the mixture dropped down below room temperature under continuous stirring, the taste-masked microparticles were formed.

FIGS. 7A-7D are FTIR spectra of starch crystal (FIG. 7A), PEG 6000 crystals (FIG. 7B), acetaminophen crystal (FIG. 7C), and the taste-masked API crystals (FIG. 7D) as shown in FIG. 8, infra, respectively.

FIG. 8 is a microscopic picture showing taste-masked API crystals which were formed by dissolving acetaminophen crystals in the heated first solution, and dissolving PEG 6000 in the heated second solution, followed by slowly adding the dissolved acetaminophen solution into the dissolved PEG 6000 solution with continuous stirring under the temperature dropped down below the room temperature.

### 4. DETAILED DESCRIPTION OF THE INVENTION

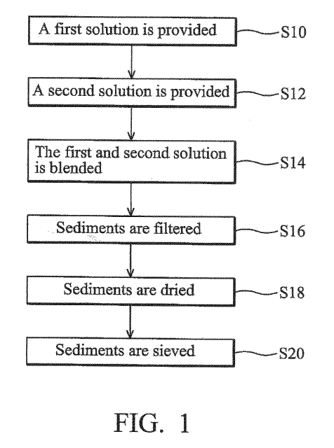

FIGS. 1-3 illustrate the method of preparing the taste-masking oral dosage form according to the embodiment of the invention. Referring to FIG. 1, a first solution is provided in step S10. The first solution comprises an API and a starch, which were dissolved in a solution, preferably water or alcohol. The uniform and viscous first solution is prepared by heating after blending.

The API may be a solute comprising a active pharmaceutical ingredient (“API”) and a starch. The solution is preferred to be water or ethanol. The API can be any orally administrated drugs, and the starch is preferred to be amylodextrin, hydroxyethyl starch, hydropropyl starch, carboxymethyl starch, acetylated starch, phosphorylated starch or a mixture thereof. The uniform and viscous first solution is obtained by heating. The temperature of the heated solution is preferred to be about or above 85° C. at least one member selected from the group:

(1) vitamins, for example, vitamin A, vitamin D, vitamin E, vitamin B₁, vitamin B₂, vitamin B₆, vitamin B₁₂, or vitamin C, minerals, for example, Ca, Mg, Fe, or protein, and amino acid or oligosaccharide and the like.

(2) antipyretic-analgesic-antiinflammatory agents, for example, aspirin, acetaminophen, ethenzamide, ibuprofen, diphenhydramine hydrochloride, dl-chorpheniramine maleate, dihydrocodeine phosphate, noscapine, methylephedrine hydrochloride, phenylpropanolamine hydrochloride, caffeine, serratiopeptidase, lysozyme chloride, tolfenamic acid, mefenamic acid, diclofenac sodium, flufenamic acid, salicylamide, aminopyrine, ketoprofen, indomethacin, bucolome, or pentazocine and the like.

(3) antipsychotic drugs, for example, chlorpromazine, reserpine, chlordiazepoxide, diazepam, imipramine, maprotiline, amphetamine, estazolam, nitrazepam, diazepam, phenobarbital sodium, scopolamine hydrobromide, diphenhydramine hydrochloride, or papaverine hydrochloride and the like.

(4) gastrointestinal function conditioning agents, for example, magnesium carbonate, sodium hydrogen carbonate, magnesium aluminometasilicate, synthetic hydrotalcite, precipitated calcium carbonate, or magnesium oxide and the like.

(5) antitussive-expectorants, for example, chloperastine hydrochloride, dextromethorphan hydrobromide, theophylline, potassium guaiacolsulfonate, guaifenesin, oxytetracycline, triamcinolone acetonide, chlorhexidine hydrochloride, or lidocaine and the like.

(6) antihistamines, for example, diphenhydramine hydrochloride, promethazine, isothipendyl hydrochloride, or dl-chlorpheniramine maleate and the like.

(7) cardiotonics, for example, etilefrine hydrochloride, procainamide hydrochloride, propranolol hydrochloride, pindolol, isosorbide, furosemide, delapril hydrochloride, captopril, hexamethonium bromide, hydralazine hydrochloride, labetalol hydrochloride, or methyldopa and the like.

(8) vasoconstrictors, for example, phenylephrine hydrochloride, carbocromen hydrochloride, molsidomine, verapamil hydrochloride, cinnarizine, dehydrocholic acid, or trepibutone and the like.

(9) antibiotics, for example, cephems, penems, carbapenems, cefalexin, amoxicillin, pivmecillinam hydrochloride, or cefotiam dihydrochloride and the like.

(10) chemotherapeutic drugs, for example, sulfamethizole or thiazosulfone and the like.

(11) antidiabetic agents, for example, tolbutamide or voglibose and the like.

(12) drugs for osteoporosis, for example, ipriflavone and the like.

(13) skeletal muscle relaxants, for example, methocarvamol and the like.

Subsequently, a second solution is prepared in step S12. The second solution comprises a solute comprising a hydrophilic polymer and optionally a surfactant. The solution is preferred to be water or ethanol. The hydrophilic polymer can be PEG, PVP, carbopol, polysaccharide, agar, MC, HPMC or a mixture thereof. The preferred hydrophilic polymer is PEG 6000. The surfactant can be any edible surfactants, preferably lecithin.

In certain embodiments, the first solution or the second solution or both solutions are heated to temperatures in the range between about 40° C. and about 99° C. and preferably between about 55° C. and about 85° C. In one embodiment, the first solution is heated to a temperature between about 80° C. and about 90° C. and the second solution is heated to a temperature between about 60° C. and about 70° C.

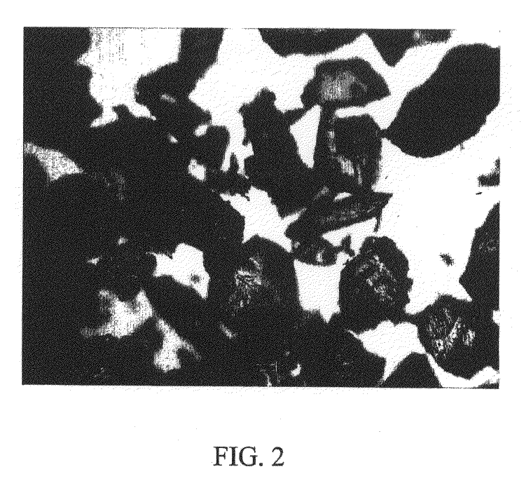

Next, the first and second solutions are blended to form a mixture in a granulating step S14. In one embodiment, the granulating step S14 comprises a wet granulating process in which the first and second solutions are mixed with continuous stirring under cooling conditions to allow the temperature of the mixture to drop below room temperature. Sediments comprising a plurality of microparticles start to form during the cooling process. FIG. 2 shows a microscopic picture of the taste-masked microparticles formed during the granulating process.

The microparticles produced in the step S14 comprise starch and taste-masked API crystals containing API masked by the hydrophilic polymer. In certain embodiments, the microparticles contain about 95% by weight of the taste-masked API crystal and about 5% by weight of starch. The taste-masked API crystal further contains API and hydrophilic polymer. The diameters of the microparticles are in the range of about 150-360 μm.

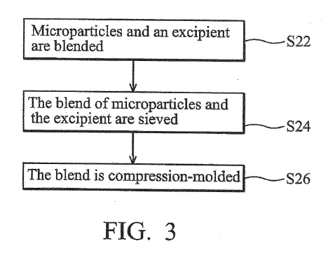

Subsequently, the sediments are filtered in step S16, dried in step S18 and sieved in step S20. Other granulating process, such as dry granulating, fluidized bed granulating, and spray granulating, may also be employed in step S14. Referring now to FIG. 3, the taste-masked microparticles produced by the process of FIG. 1 are blended with excipients in step S22. The excipients comprises disintegrating agents, effervescent agents, sweeteners, and lubricants comprising saccharide, alcohol, and sugar alcohol, wherein saccharide comprises monosaccharide or disaccharide, and sugar alcohol comprises mannitol, sorbitol, xylitol, or glycerol.

Subsequently, the mixture of the taste-masked microparticles and the excipients is sieved in step S24. After sieving, the mixture is compression-molded in step S26 with a tabletting machine, for example, a High-Speed Rotary Tabletting Machine.

The molding pressure of the High-Speed Rotary Tabletting Machine is about 800˜1200 lb/cm², and preferably 1000 lb/cm². The molding speed thereof is about 15˜20 rpm, and preferably 16 rpm.

The taste-masking oral dosage form of the invention comprises an API which is about 35˜45% by weight, a starch which is about 20˜30% by weight, a hydrophilic polymer which is about 2˜10% by weight, a surfactant which is about 2˜10% by weight, and other excipients which are about 40˜50% by weight. Additionally, the porosity of the tablet is about 30˜70%, the disintegration time (the time required to complete dissolution by saliva in an oral cavity in a healthy adult male) thereof is less than 1 min, the hardness thereof is about 20˜50NT, and the brittleness thereof is less than 2%.

The following experimental designs and result are illustrative, but not limiting the scope of the present invention. Reasonable variations, such as those occur to reasonable artisan, can be made herein without departing from the scope of the present invention. Also, in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### Example 1

### Production of Taste-Masked Microparticles

A first solution comprising acetaminophen (antipyretic-analgesic-antiinflammatory agents), amylodextrin, and H₂O was prepared as described in the following steps. First, 400 g of acetaminophen and 84 g of amylodextrin were added into 1600 ml of H₂O, stirred, and the first solution was heated to 90° C. No crystalline structure was found precipitating out of the first solution even when the solution was cooled to a temperature below room temperature.

A second solution comprising a PEG 6000 (hydrophilic polymer), lecithin (surfactant), and H₂O was prepared as the following step. First, 50 g of PEG 6000 and 50 g of lecithin were added into 790 ml of heated H₂O at 70° C., and stirred to dissolve completely. No crystalline structure was found precipitating out of the second solution when it was cooled to a temperature below room temperature.

Next, a crystallization process was performed, wherein the first solution was slowly added into the second solution, and continuously stirred in a cooling environment. Sediments started to form after the temperature of the mixture of the first and second solutions dropped below room temperature. The sediments were filtered through a Buchner funnel, and dried in a dryer at 45° C. Then, the sediments were sifted through a sieve with 400 μm diameter mesh.

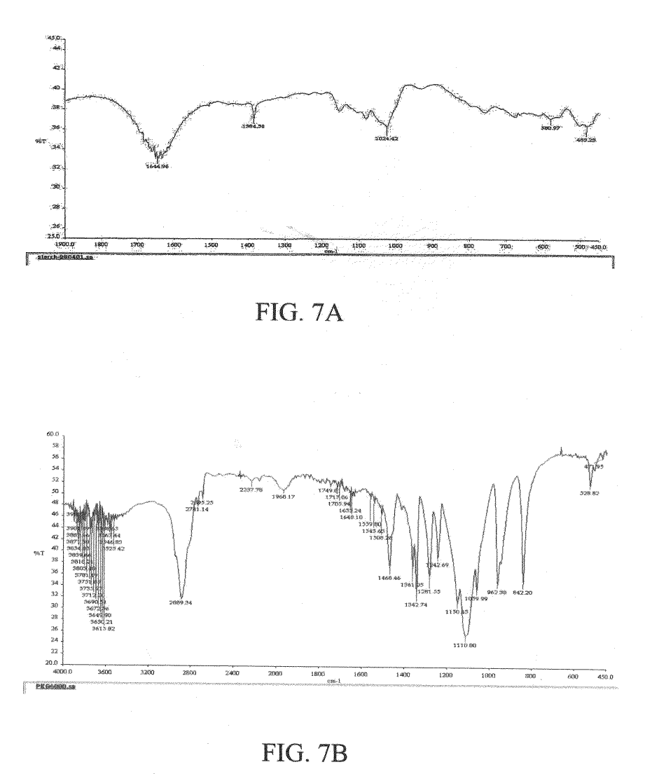

The sediments have no taste, suggesting that the bitter taste of acetaminophen has been masked. Microscopic examination of the sediments showed the taste-masked microparticles with a crystalline structure and had a diameter ranging between about 150 and 360 μm. The crystalline structure was polyhedron in shape (FIG. 6) and could not be stained with iodine, suggesting that the taste-masked microparticles were not covered by starch (FIG. 6).

A further analysis of the content of the taste-masked microparticles confirmed that ≧95% by weight of the microparticles were acetaminophen, indicating that the taste-masked microparticles comprised predominantly the API.

Next, microparticles and excipients were blended with a V-shaped blender. The excipients comprises 250 g of lactose (disaccharide), 100 g of mannitol (sugar alcohol), and 100 g of crospovidone (disintegrating agents). Subsequently, the blend was sifted through a sieve with 200 μm diameter mesh. Finally, 400 g of the blend was compression-molded to form a tablet with a High-Speed Rotary Tabletting Machine. The molding pressure was about 1000 lb/cm², and the molding speed was about 15.9 rpm.

In this example, 400 g of acetaminophen is about 40% by weight, 50 g of amylodextrin is about 5% by weight, 50 g of PEG 6000 is about 5% by weight, 50 g of lecithin is about 5% by weight, and 450 g of excipients comprising 250 g of lactose, 100 g of mannitol, and 100 g of crospovidone are about 25% by weight of the taste-masking oral dosage form.

### Example 2

### Production of Taste-Masked API Crystals

Because the taste-masked microparticles contained predominantly the API, a further investigation into whether the crystalline-structure found in the taste-masked microparticles represented a new form of API crystal or was a co-crystal of the API and the starch was conducted as follows:

A first solution was prepared containing about 400 g of acetaminophen and about 1600 ml of H₂O. The first solution was stirred and heated to about 85° C. No crystalline structure was found precipitating out of the first solution even when the solution was cooled to a temperature below room temperature.

A second solution containing about 50 g of PEG 6000 (hydrophilic polymer) and about 790 ml of H₂O was prepared. The second solution was heated to about 60° C. with stirring until PEG 6000 was completely dissolved. No crystalline structure was found precipitating out of the second solution when it was cooled to a temperature below room temperature.

Next, a crystallization process according to FIG. 1 was performed, wherein the first solution was slowly added into the second solution, and continuously stirred in a cooling environment. Sediments started to form after the temperature of the mixture of the first and second solutions dropped below room temperature. The sediments were filtered through a Buchner funnel, and dried in a dryer at 45° C. Then, the sediments were sifted through a sieve with 400 μm diameter mesh.

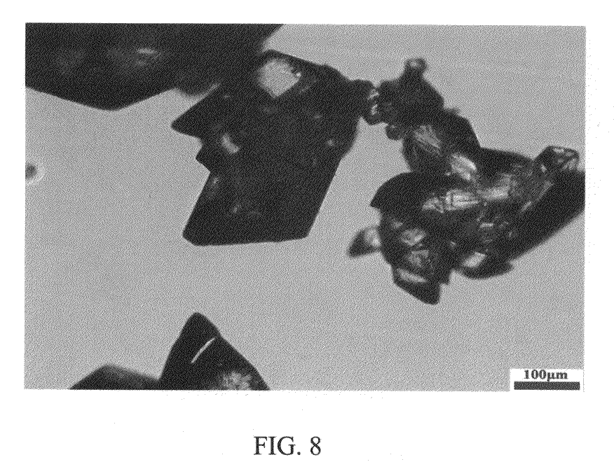

The sediments did not have bitter taste, suggesting that the bitter taste of acetaminophen has been masked by PEG. Microscopic examination of the sediments showed a crystalline-structure which was polyhedron in shape and had a diameter ranging between about 150 and 360 μm (FIG. 8). A close comparison of the microscopic pictures of the taste-masked microparticles in FIG. 6 and the taste-masked API crystal in FIG. 8 indicated that they were similar. The only major difference between the two was that the taste-masked microparticles had no taste while the taste-masked API crystals were predominantly tasteless. This indicated that the taste-masked microparticles might contain a small quantity of starch either separated from the taste-masked API crystals or incorporated into the taste-masked API crystals.

### Example 3

### Production of Starch Microspheres

To investigate the involvement of the starch in the formation of the taste-masked API crystals, a separate study of the starch during the crystallization process disclosed in FIG. 1 was conducted.

A first solution containing about 84 g of amylodextrin and about 1600 ml of H₂O was prepared. The first solution was stirred and heated to about 85° C. No crystalline structure was found precipitating out of the first solution even when the solution was cooled to a temperature below room temperature.

A second solution containing about 50 g of PEG 6000 (hydrophilic polymer) and about 790 ml of H₂O was prepared. The second solution was heated to about 60° C. with stirring until PEG 6000 was completely dissolved. No crystalline structure was found precipitating out of the second solution when it was cooled to a temperature below room temperature.

Next, a crystallization process according to FIG. 1 was performed, wherein the first solution was slowly added into the second solution, and continuously stirred in a cooling environment. Sediments started to form after the temperature of the mixture of the first and second solutions dropped below room temperature. The sediments were filtered through a Buchner funnel, and dried in a dryer at 45° C. Then, the sediments were sifted through a sieve with 400 μm diameter mesh.

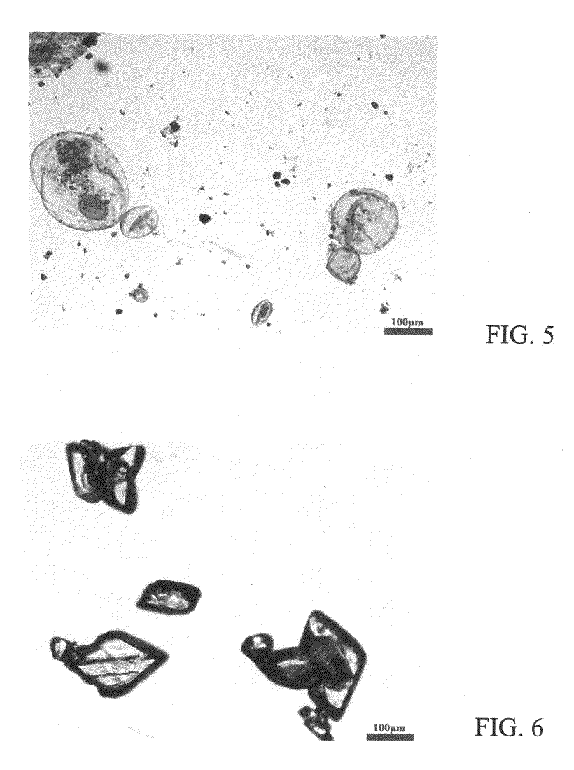

The sediments were analyzed under the microscope and further stained with iodine. As shown in FIG. 5, round, sac like microspheric structures were found in the sediments. These microspheres could be stained with iodine, indicating that they were starch microspheres. The diameters of the starch microspheres ranged between 50 μm and 250 μm. Clearly, these starch microspheres were different from those of the taste-masked API crystals or the taste-masked microparticles found in Examples 1 and 2.

### Example 4

### Morphological and FTIR Spectral Analysis of the Taste-Masked Microparticles of Example 1

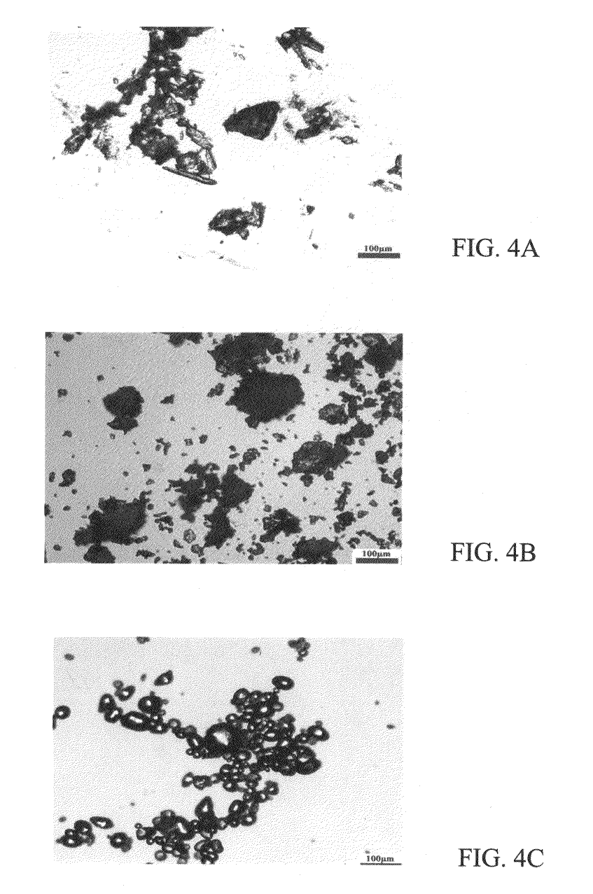

FIGS. 4A-4C are microscopic pictures showing respective crystalline structure of acetaminophen (FIG. 4A), PEG 6000 (FIG. 4B) and starch (FIG. 4C) before the crystallization process according to FIG. 1 took place. As shown in FIG. 4A, the acetaminophen crystals were tubular or needle-like structures with the longitudinal side ranged between 20 μm to 150 μm.

As shown in FIG. 4B, PEG 6000 crystals appeared to be amorphous structure forming aggregates with size ranged between 10 μm and 150 μm.

As shown in FIG. 4C, the starch were round- or oval-shaped small particles with sizes around 20-50 μm in diameter. This was in contrast with the starch microspheres prepared in Example 3, as shown in FIG. 5.

As shown in FIG. 6, the taste-masked microparticles prepared in Example 1 contained crystals with polyhedron shape. The taste-masked microparticles were distinctively different from the acetaminophen crystals (FIG. 4A), PEG crystals (FIG. 4B), and the starch crystals (FIG. 4C), respectively, without the crystallization process. The taste-masked microparticles were also distinctively different from the starch microspheres shown in FIG. 5.

Also, because the taste-masked microparticles of FIG. 6 could not be stained with iodine, the starch microspheres might not be present on the surface of the microparticles.

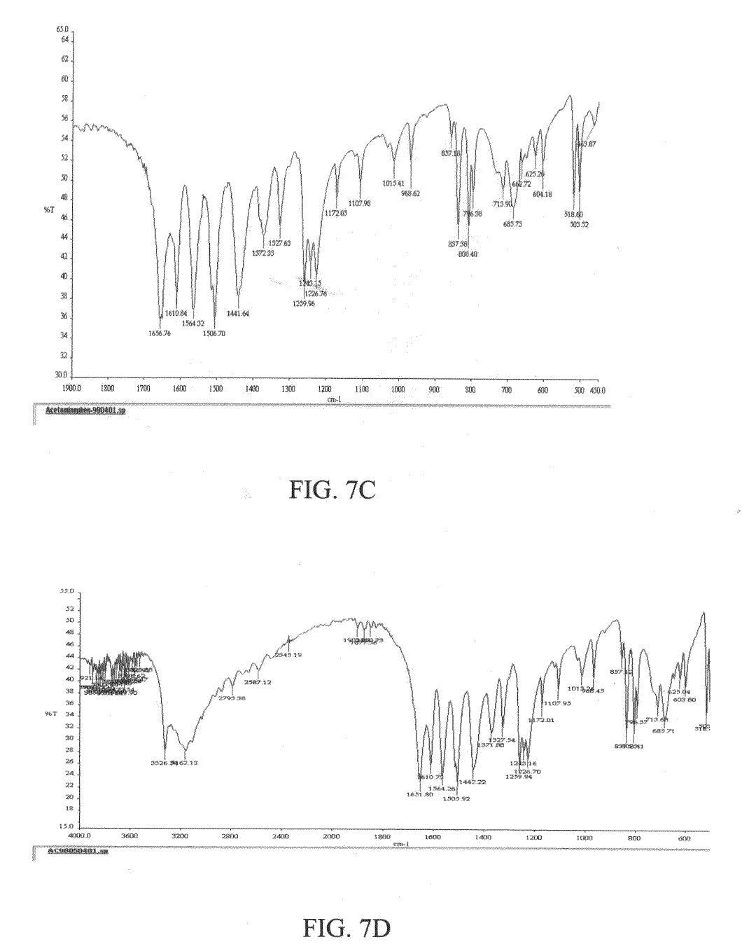

To further analyze the structure of the taste-masked microparticles prepared in Example 1, the taste-masked microparticles were further analyzed by FTIR. As shown in FIGS. 7A-7D, the respective IR spectrum of the starch crystal (FIG. 7A), the PEG 6000 crystal (FIG. 7B), the acetaminophen crystal (FIG. 7C), and the taste-masked API crystals prepared in Example 2 (FIG. 7D) were analyzed.

As shown in FIG. 7A, the starch contained 5 wavelength peaks at 483.25 cm⁻¹, 580.97 cm⁻¹, 1024.42 cm⁻¹, 1384.58 cm⁻¹, and 1644.96 cm⁻¹.

As shown in FIG. 7B, the PEG6000 contained 25 wavelength peaks between 400 cm⁻¹ and 3000 cm⁻¹, which are 471.95 cm⁻¹, 528.82 cm⁻¹, 842.20 cm⁻¹, 962.38 cm⁻¹, 1059.99 cm⁻¹, 1110.80 cm⁻¹, 1150.45 cm⁻¹, 1242.89 cm⁻¹, 1281.55 cm⁻¹, 1342.74 cm⁻¹, 1361.05 cm⁻¹, 1468.48 cm⁻¹, 1508.26 cm⁻¹, 1543.63 cm⁻¹, 1559.80 cm⁻¹, 1648.10 cm⁻¹, 1655.24 cm⁻¹, 1705.94 cm⁻¹, 1717.86 cm⁻¹, 1749.81 cm⁻¹, 1968.17 cm⁻¹, 2297.78 cm⁻¹, 2693.25 cm⁻¹, 2741.14 cm⁻¹, 2889.34 cm⁻¹. None of the peaks shown in the PEG6000 were found in the starch crystals, indicating the dissimilarity between the two crystals.

As shown in FIG. 7C, the acetaminophen crystals contained the following wavelength peaks between 500 cm⁻¹ and 1700 cm⁻¹: 503.52 cm⁻¹, 518.60 cm⁻¹, 604.18 cm⁻¹, 625.20 cm⁻¹, 662.72 cm⁻¹, 685.73 cm⁻¹, 713.90 cm⁻¹, 796.38 cm⁻¹, 808.40 cm⁻¹, 837.38 cm⁻¹, 857.18 cm⁻¹, 968.62 cm⁻¹, 1015.41 cm⁻¹, 1107.98 cm⁻¹, 1172.05 cm⁻¹, 1226.76 cm⁻¹, 1243.15 cm⁻¹, 1259.96 cm⁻¹, 1327.63 cm⁻¹, 1372.33 cm⁻¹, 1441.64 cm⁻¹, 1506.70 cm⁻¹, 1564.32 cm⁻¹, 1610.84 cm⁻¹, and 1656.76 cm⁻¹. None of these peaks was in common with either the starch or the PEG6000.

However, as shown in FIG. 7D, which was the taste-masked API crystals prepared in Example 2, the wavelength peaks between 450 and 1700 include the following: 503.14 cm⁻¹, 518.40 cm⁻¹, 603.80 cm⁻¹, 625.04 cm⁻¹, 685.71 cm⁻¹, 713.68 cm⁻¹, 796.37 cm⁻¹, 808.41 cm⁻¹, 838.08 cm⁻¹, 857.12 cm⁻¹, 1015.26 cm⁻¹, 1107.93 cm⁻¹, 1172.01 cm⁻¹, 1226.70 cm⁻¹, 1243.16 cm⁻¹, 1259.94 cm⁻¹, 1327.54 cm⁻¹, 1371.88 cm⁻¹, 1442.22 cm⁻¹, 1505.92 cm⁻¹, 1564.26 cm⁻¹, and 1651.80 cm⁻¹. The only significantly different peak between the acetaminophen crystals and the taste-masked API crystals was peak 1656 cm⁻¹ (showing the carbonyl group stretching) of the acetaminophen crystals that were not in the taste-masked API crystals, suggesting that the carbonyl group of acetaminophen interacted with PEG 6000 to form hydrogen bonding.

This further supports the findings that although the crystalline structure of the taste-masked API crystals was significantly different from those of the API crystals (Cf 4A and 8), the predominant content of the taste-masked API crystals was still the API, even though the bitter taste of the API was masked.

### Example 5

### Characteristics of Tablets Prepared Using the Microparticles of Example 1

To illustrate the effects of the invention in further detail, the following characteristics of the tablets prepared in the foregoing example was determined, comprising disintegration time, hardness, and brittleness. The results are shown in Table 1.

### (1) Disintegration Time

The disintegration time of each tablet was determined in accordance with the disintegration test as described in the following. First, 37±2° C., proper amount of water used as solvent was added into the container of the test machine (PHARMA TEST PTZ1 E type). Next, six tablets were added into the container, and the container was covered by a plastic cover. Subsequently, the test machine shook the container until the tablets were disintegrated completely. The mean of the results of six determinations of each API was adopted respectively.

### (2) Hardness

The hardness of each tablet was determined in accordance with the hardness test as described in the following. First, six tablets were placed on the hardness tester (SHIN KWANG SK-32060 type). Next, pressure was applied from the long axis until the tablets were cracked. The mean of the results of six determinations of each API was adopted respectively.

### (3) Brittleness

The brittleness of each tablet was determined in accordance with the brittleness test as described in the following. First, 6-6.5 g of sixteen tablets (380-420 mg/per tablet) were placed on the sieve (10 mesh). After dropped powders were removed, the precise sample weight (As) was measured. The sample was then added into the test machine (PHARMA TEST PTFE type), and the test machine spun at a speed of 25 rpm for 100 turns. After the sample was taken out, all dropped powders were removed again. Finally, the precise sample weight (A₀) was measured. As a result, Brittleness=(A₀/As)*100. The mean of the results of sixteen determinations of each active pharmaceutical ingredient was adopted respectively.

TABLE 1

| Active | | | |
|---|---|---|---|
| pharmaceutical | Disintegration | Hardness | brittleness |
| ingredient | time (sec) | (NT) | (%) |
| acetaminophen | 20 ± 5 | 24.9 ± 7.0 | 1.2 |

The results of Table 1 indicate that the disintegration time of the oral dosage forms of the present invention is less than 1 min, and the brittleness thereof is less than 2%. Therefore, the elderly, children, or those with impaired swallowing ability are able to swallow the tablets, due to rapid disintegration and absorption in an oral cavity. Additionally, an adequate mechanical strength of 20˜50NT is obtained, facilitating the packaging process in production lines.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A taste-masked microparticle comprising:
active pharmaceutical ingredient (API) crystal masked by a hydrophilic polymer to form a taste-masked API crystal; and
a starch
wherein said taste-masked API and said starch are packaged together to form said taste-masked microparticle.

2. The taste-masked microparticle according to claim 1, wherein said taste-masked API crystal is morphologically different from said API crystal.

3. The taste-masked microparticle according to claim 1, wherein said microparticle comprises ≧95% by weight of said taste-masked API crystal.

4. The taste-masked microparticle according to claim 1, wherein the hydrophilic polymer is at least one selected from group consisting of PEG, PVP, carbopol, polysaccharide, agar, MC, and HPMC.

5. The taste-masked microparticle according to claim 1, wherein the hydrophilic polymer is polyethylene glycol.

6. The taste-masked microparticle according to claim 5, wherein the polyethylene glycol is PEG 6000.

7. The taste-masked microparticle according to claim 1, wherein the API is an orally administrated drug.

8. The taste-masked microparticle according to claim 8, wherein the API has bitter taste and wherein the taste-masked API crystal does not have bitter taste.

9. The taste-masked microparticle according to claim 1, wherein the API is acetaminophen and wherein the hydrophilic polymer is PEG 6000.

10. The taste-masked microparticle according to claim 9, wherein the taste-masked API crystal is polyhedron in shape.

11. The taste-masked microparticle according to claim 1, wherein the starch is at least one selected from the group consisting of amylodextrin, hydroxyethyl starch, hydropropyl starch, carboxymethyl starch, acetylated starch, and phosphorylated starch.

12. The taste-masked microparticle according to claim 1, wherein the taste-masked microparticle has a diameter in the range between about 150 μm and about 350 μm.

13. A taste-masking oral dosage form comprising said taste-masked microparticle according to claim 1 and an excipient.

14. The taste-masking oral dosage form according to claim 13, wherein said taste-masking oral dosage form is a tablet.

15. The taste-masking oral dosage form according to claim 1, wherein said taste-masking oral dosage form further comprises a surfactant.

16. The taste-masking oral dosage form according to claim 15, wherein the surfactant is an edible surfactant.

17. The taste-masking oral dosage form according to claim 15, wherein the surfactant is a lecithin.

18. The taste-masking oral dosage form as claimed in claim 15, wherein the surfactant comprises lecithin.

19. The taste-masking oral dosage form according to claim 13, wherein the excipient is at least one selected from the group consisting of disintegrating agents, effervescent, lubricants, sweeteners, saccharide, alcohol, and sugar alcohol.

20. The taste-masking oral dosage form according to claim 19, wherein said saccharide is monosaccharide or disaccharide.

21. The taste-masking oral dosage form according to claim 19, wherein the sugar alcohol is at least one selected from the group consisting of mannitol, sorbitol, xylitol, and glycerol.

27. The taste-masking oral dosage form as claimed in claim 14, wherein the porosity of the tablet is about 30˜70%.

28. The taste-masking oral dosage form as claimed in claim 13, wherein the brittleness of the tablet is less than 2%.

29. A taste-masked active pharmaceutical ingredient (API), comprising an API with a bitter taste and a hydrophilic polymer, wherein the hydrophilic polymer masks the bitter taste of the API.

30. The taste-masked API of claim 29, wherein the API and the hydrophilic polymer form a crystalline structure.

31. The taste-masked API of claim 29, wherein the API is acetaminophen and wherein the hydrophilic polymer is PEG 6000.
